(19) United States
(12) Patent Application Publication
(10) Pub. No.: US 2017/0140120 A1
(43) Pub. Date: May 18, 2017

(54) MOBILE DATA HUB FOR PATIENT MONITORS

(71) Applicant: Shenzhen Mindray Bio-Medical Electronics Co., Ltd., Shenzhen (CN)

(72) Inventor: James Patrick Thrower, Oakland, NJ (US)

(21) Appl. No.: 14/943,831

(22) Filed: Nov. 17, 2015

(51) Int. Cl. G06F 19/00; A61B 5/00

(52) U.S. Cl. CPC G06F 19/3418; A61B 5/6898; A61B 5/0077; G06F 19/323; G06F 19/321

(57) ABSTRACT

A mobile device such as a smart phone or tablet computer is paired with a patient monitor. Data is then transferred between the two wirelessly and, via the mobile device, may also be uploaded to a remote server. The data may include annotation and comments, in either text or audio form, as well as executable code and images.

### TECHNICAL FIELD

This disclosure relates to patient monitoring and, in particular, to an arrangement and method of operation for data transfer to and from a patient monitor.

### BACKGROUND

Information management has become as important in the context of patient care as it is in so many other aspects of modern life. Patient information is of many types and comes from many sources. For example, patient monitors are routinely used to gather and present data from sensors measuring blood pressure, SpO2, temperature, ECG, and other vital signs. Paper or electronic charts and records keep track of such identifying and treatment information as the patient's name, physician's notes and directions, medication schedules, etc. Sometimes, the patient monitor is used to store some or all of the identifying and treatment information as well. The patient monitor itself generates and requires data relating to itself, such as its software version number, status information, which sensor modules are connected, and the code that controls and defines its operation.

Most patient monitoring still relies on data input and management via keyboards, touch screens, and even paper and pen, all of which increase the complexity of the devices involved and inconvenience to their users. All interaction between users and a patient monitor requires some form of hardware and software, and as the monitors become smaller, it becomes more challenging to input information through built-in interfaces. One other problem of existing monitor-user interfaces is that they are essentially monitor-centered: the user must adjust to the interface design of the monitor, which is typically different from manufacturer to manufacturer and usually even from product to product by the same manufacturer. What is needed is a system that reduces the data I/O burden in the context of patient monitoring.

### BRIEF DESCRIPTION OF THE DRAWINGS

FIG. 1 illustrates the main components of a system in which a mobile device is used for data communication with a medical device such as a patient monitor.

FIG. 2 illustrates hardware and software modules within the components shown in general in FIG. 1.

FIG. 3 illustrates data storage within the components shown in general in FIG. 1.

### DETAILED DESCRIPTION

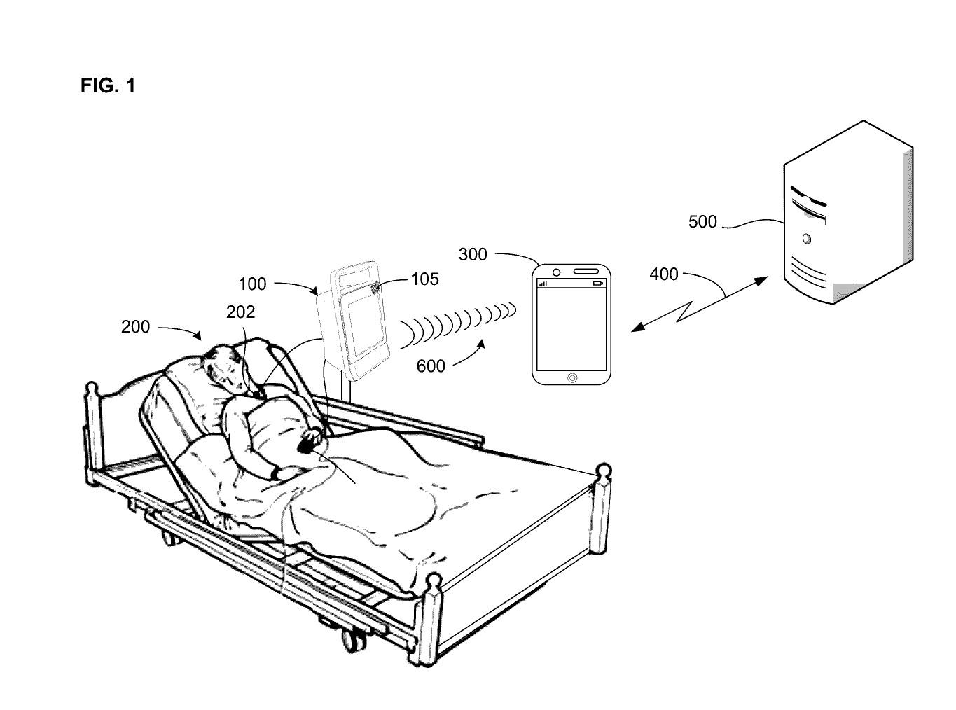

FIG. 1 illustrates the general context in which embodiments will typically be used: A medical device 100 is paired and associated with a patient 200, typically receiving inputs from any of a number of sensors 202. Below, the medical device is exemplified as a patient monitor 100. Embodiments may be used with other devices as well, however. For example, many modern diagnostic systems such as imaging devices (ultrasound, MRI, etc.), treatment systems (anesthesia machines, etc.), and even more generalized systems such as central stations or workstations that are used to monitor patients or review data receive user input and may benefit from the features of embodiments described below.

A mobile device 300 communicates with the monitor 100, preferably using any common wireless technology such as Bluetooth, Near Field Communication (NFC), radio frequency or optically (including infrared) encoded signals, etc. Although wireless communication between the devices 100, 300 will in most cases be most convenient, for some implementations, it would also be possible to connect the two at least temporarily using wired means such as cables with standard USB connectors.

It is known in the field of telemedicine for individual, dedicated patient sensors to send their signals to a mobile device for remote processing. In the context of embodiments here, however, it is assumed that the medical device is a multi-function monitoring device able to receive and process patient data acquired from sensors.

Any preferred mobile device 300 may be used in different embodiments. One advantage of this is that users such as physicians and nurses may use devices that they are familiar with and in fact probably already have with them as they attend to the patient 200. For example, the mobile device 300 may be a smart phone or a tablet computer. In these cases, given the features found in most modern smart phones and tablets, embodiments may be implemented as an application loaded into the device 300, with no need for hardware modification or specialization. In general, a “mobile device” in this context are any user-portable devices that have features of a modern “smart phone” or tablet computer, including the ability to execute code (such as the code defining the various software modules described below) on one or more processors, to store data, to pair and exchange data with the patient monitor, to accept input in any desired format (binary, textual, audio, even visual, etc.) from a user and/or external source and/or from internally generated routines, to communicate information to the user in textual and/or graphic and/or audible form, and to communicate via one or more networks with the external sources depending on which features are desired.

In order to transfer information between the two, the mobile device 300 will need to establish communication with the monitor 100 using any known protocol; in other words, they should be “paired.” This may be done using the same technology as for data transfer itself, for example, NFC, Bluetooth, etc., or one technology may be used to establish pairing while another is used for data transfer after pairing is completed. Pairing may be done in a more manual manner, for example by scanning a mark 105 placed on the monitor 100 either at the time of manufacture or later, for example, by the owner. The mark 105 need not be physical, but could also be presented on a display 150. The mobile device and patient monitor could also pair by the mobile device sensing, via a microphone 360, an audio signal emitted by the monitor, via a tone generator, speaker unit 136, etc. If technologies such as Bluetooth are incorporated to enable such peer-to-peer connection, the mobile device 300 may also be set to sense when it is within a pairing range of the monitor 100, and to pair automatically after an initial pairing. Pairing may thus be substantially automatic, for example, as soon as the two devices are within signal range of each other, or at least partially manually, for example, by having the user using the device 300 scan the marking 105. It would also be possible for the mobile device 300 to use locational awareness such as by GPS positioning, and to initiate communication when within a given distance of the monitor 100. All such methods would allow the mobile device 300 to connect with the patient monitor 100 and to operate even in the absence of a dedicated network.

In general, “pairing” refers to the procedure by which the mobile device and the patient monitor establish a communications link between each other. Depending on the implementation, this may be all that is needed. In other cases, however, the patient monitor and the mobile device will need to be associated specifically with respect to their identities (such as, for example, patient monitor serial number and mobile device phone number), possibly together with the ID of the patient being monitored. In other words, logical and/or administrative association may be implemented in addition to technical pairing. In some cases, the technical pairing itself may suffice to associate the mobile device logically with a patient monitor. In other implementations, the mobile device and patient monitor may exchange additional data (input by the user, stored by an administrative system, generated by an application, etc.) to establish the logical association, which may be done either manually or automatically. Such logical association may also be done indirectly, by the mobile device querying a remote administrative system, such as a server 500.

In some embodiments, depending upon how extensive the information is that is to be exchanged between the mobile device 300 and the monitor 100, what type of information is to be exchanged, and what the purpose is, these two devices may be sufficient. In other embodiments, however, the primary intended communication path may be between the monitor 100 and a remote, either physical or virtual, system, such as the server 500. In such cases, the mobile device 300 may be used as an intermediary, thereby reducing the need to incorporate additional networking capability into the monitor 100.

In another embodiment, the mobile device 300 may log into the central server 500 through any known connection protocol 400, which may be the standard cellular telephone network, Wi-Fi via a router, or any other wireless technology. The mobile device 300 may then be paired with the monitor 100 through either central assignment by the server 500 or by other means such as entering an identification number for the monitor 100, scanning the optical marker 105, etc. Especially given the context of I/O of patient information, any data transmitted between the mobile device and the server 500 preferably encrypted, especially any data specific to the patient and any data transmitted over a non-dedicated, public network. Encryption may also be used for patient-related or other data transmitted between the mobile device and the patient monitor.

The general operating principle of embodiments is that a user may enter data via the mobile device 300 into the patient monitor or other medical device 100. Any type of data could be transferred. For example, a doctor could dictate observations or instructions into his phone, whose existing voice recognition software could convert it to a form suitable for transfer to the patient monitor 100 and appropriate storage, or it could be transferred without conversion to text, as an audio file, which may then be replayed by the monitor 100 if it is equipped with an appropriate speaker 136. The data being transferred may also be commands, generated by the mobile device itself, such as via an installed application, or transcription data. The voice recognition software may be local, that is, running in the mobile device 300 itself, or it could be remote and accessible via a network. Using the physical or virtual keypad of the mobile device 300, a user could also enter values that, upon transmission to the monitor and interpretation by its software, adjust settings such as which information is to be displayed or other monitoring and control parameters.

Data to be transferred from the mobile device 300 to the patient monitor 100 could also come from another source, even separate from the server 500. For example, assume that a patient is being monitored but is temporarily moved to a different unit for some other procedure, such as an imaging scan or other tests. The results of the scan or tests could thereby be loaded into the mobile device 300, then transferred into the monitor 100 so that all the information could be available to the attending physician, in the monitor 100, in one place.

It is not necessary for the data transmission to be one way, from the mobile device 300 to the patient monitor 100. Rather, the patient monitor 100 could also transfer data to the mobile device 300. For example, a nurse or doctor could upload current or even trend values from the monitor 100 into her phone, which could then display them for viewing and analysis even when she is away from the patient 200. Moreover, such uploaded data could then be transferred onward by uploading to the central server 500 for appropriate storage or viewing and analysis there, such as in a telemedicine context. In such cases, the mobile device 300 may function as an intermediate collection and data transfer hub, such that it would not be necessary to include such circuitry and software within the monitor 100 itself. As one of many possible examples of a use for this capability, paramedics could upload into a mobile device such as a tablet the monitoring data of a patient en route, including, for example, a “cine” recording showing the values captured by the monitor 100 during the entire transit time, and this data could be easily downloaded into a computer or even other monitor in the emergency room.

The data transferred into the monitor 100, or uploaded to another system such as the server 500, need not be alphanumeric; it could, for example, be visual. For example, if the mobile device 300 is equipped with a camera, such as the same one used to capture the marking 105, then the user could also use it to capture images of, for example, some aspect of the body of the patient 200, or of the placement of sensor electrodes. These images could then also be transferred into the monitor 100 for storage and later viewing, and/or uploaded to the server 500.

It is also not necessary for the data transferred between the devices 100, 300 to be “passive.” Rather, it would also be possible to transfer executable code from the mobile device 300 to the monitor 100. In these cases, the mobile device 300 may function as a kind of downloading portal for, for example, upgraded software, which is then downloaded into the patient monitor 100 using techniques known even now for downloading software and upgrades into mobile devices themselves. In such an embodiment, the system software of the monitor 100 may load and subsequently execute the code using known techniques. Such an ability would reduce the current need to remove the monitor 100 to perform software upgrades, which may be performed when convenient for the user and not necessarily dependent on the normal product cycle of the monitor 100. By going from monitor to monitor, it would thereby also be possible to upgrade the software of several monitors 100 simply by pairing the mobile device 300 with each in turn and performing the code transfer via a wireless, paired connection 600.

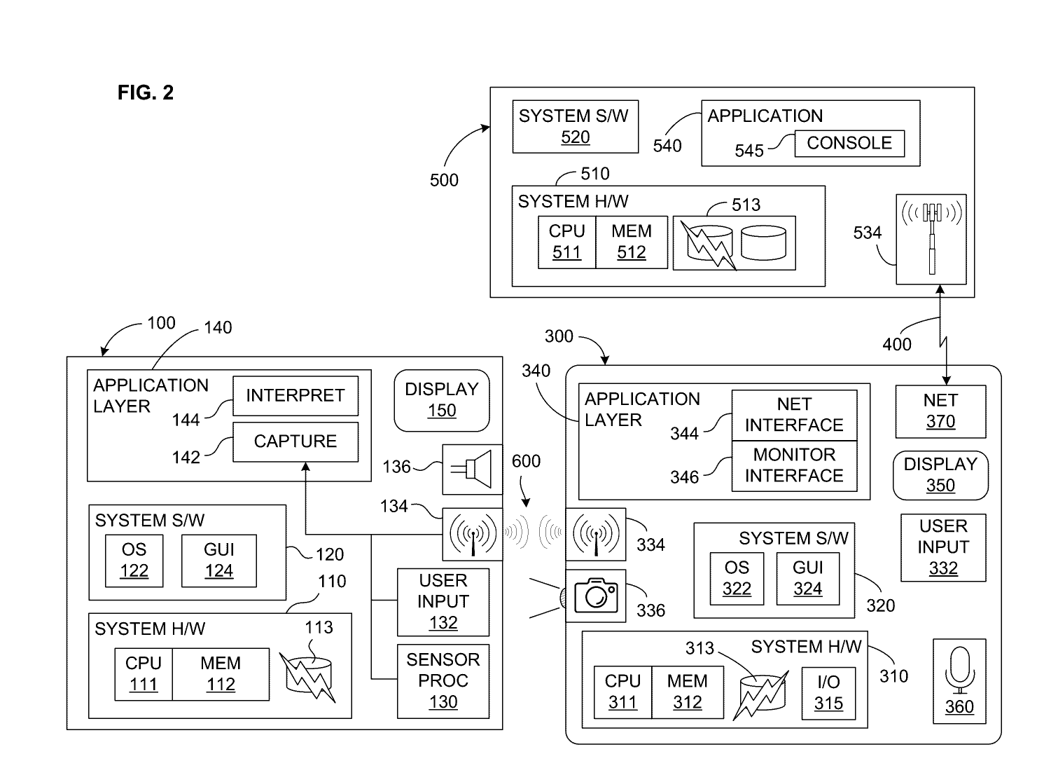

FIG. 2 illustrates the software and hardware components that may be included in embodiments of the patient monitor 100, the mobile device 300, and, if included in the implementation, the remote server 500. The monitor 100 will typically include some version of system hardware 110, including one or more central processors 111, volatile memory 112, and some form of non-volatile storage 113, which may be implemented using any known technology, from mechanically addressable hard disks to flash and similar solid-state storage devices. System software 120 such as an operating system 122 or equivalent, as well as some form of graphical user interface software 124, will also be included in a typical patient monitor 100.

Sensor processing devices and software, such as respective drivers, are shown collectively as the component 130. In typical patient monitors, some form of connector and dedicated processing module is usually plugged into the monitor to condition its respective sensor signals for processing by the monitor and proper display. The monitor 100 may also include user input devices 132 such as a keyboard, touchscreen, knobs, and buttons. The data generated by whichever sensors and user input devices are included in any given implementation may be processed and interpreted either by system software or by application-level software such as a data capture module 142 and a software module 144, which interpret the data and process it into a form suitable for presentation on the display unit 150 and/or as sound, via the speaker unit 136. The monitor 100 will also include a transceiver 134 suitable to whichever wireless connection it is to make with the mobile device 300. For example, the transceiver 134 could be Bluetooth, NFC, or radio frequency circuitry, or any combination of these.

As in mobile devices, the mobile device 300 will similarly include system hardware 310 and software 320, with one or more processors 311; memory devices 312; persistent storage 313, such as an SD card or flash memory; and I/O control circuitry 315. System software 320 of the mobile device 300 will similarly include some form of operating system 322 and software that controls the graphic user interface 324. A transceiver 334 is included to communicate with the monitor 100 using any architected technologies such as Bluetooth, NFC, or optical signals.

In embodiments in which the user pairs the mobile device 300 with a patient monitor 100 by scanning an optical marker 105, for example, a QR code, barcode, alphanumeric marking, or even a suitable display on the screen of the monitor 100, a built-in camera 336 of the mobile device 300 may be used. Optical scanning of such markers and interpretation of the data they encode is well known and may be used. Application-level software/layer 340 will generally include the software used to connect the device to one or more networks, such as the telephone network, the Internet, or a local area network, as well as monitor interface software 346 used to format and transfer data to the monitor 100. The mobile device 300 will also include various user input devices 332 such as a virtual or physical keypad, touch screen, and various buttons and switches. The mobile device 300 will also include a display 350 and a microphone module 360, which will be used in embodiments in which a user is allowed to speak information that is to be transmitted to the monitor 100. The illustrated microphone module 360 may include not only the physical microphone itself, but also the circuitry and software needed for A/D conversion and, if included, speech recognition. In some embodiments, instead of having built-in speech recognition capability, the mobile device may instead submit audio (voice) data to a remote speech-recognition application located in a separate system, including even otherwise unrelated, generally accessible, cloud- or Internet-based routines.

The mobile device 300 will also include whatever network connection circuitry and drivers 370 are needed to communicate with the server 500 over whatever network 400 is used, such as the telephone network or a local wireless network connection. Note the network over which the mobile device communicates with the server 500 need not, and, in most anticipated cases, will not be the same as the network or communications link over which the mobile device and the patient monitor exchange data. Indeed, the server may not even be accessible to the patient monitor, although this is not a requirement of any embodiment. Having the mobile device handle communication with remote systems such as the server 500, services, and information not available to or shared with the patient monitor itself, however, allows for a wide range of capabilities even with patient monitors that may not have or be configured for network access, which also simplifies the monitors themselves.

In embodiments in which data transfer is to occur between the mobile device 300 and the remote server 500, such as an administrative server, for example, to enable data transfer between the server 500 and the monitor 100, the server 500 will also include hardware 510 and software 520, such as one or more processors 511, memory devices 512, and storage devices 513. Application-level software 540 may also be included in the server 500. In embodiments in which a user is to access data in the server 500, for example, to view data transferred from one or more monitors 100 or to enter commands to be transferred to those monitors 100, the server 500 may include any software to present a console 545 to the user, in which case input and output (I/O) devices will also be included in the server 500. The server 500 may also include whatever network transceiver 534 is needed to communicate with the mobile device 300.

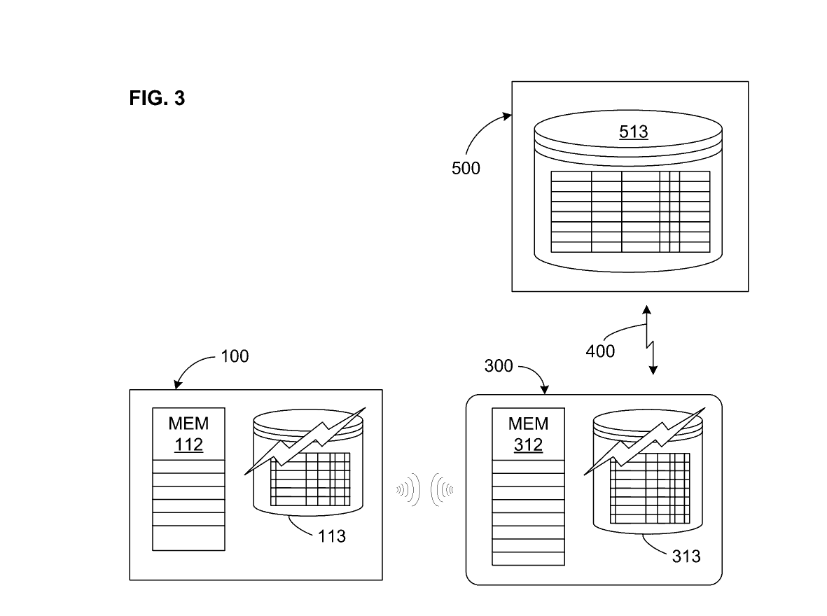

FIG. 3 illustrates how data indexed and stored within the monitor 100 may be transferred to the mobile device 300, optionally for further transfer to the central server 500. For example, the patient monitor data may be structured to be associated with the patient currently being monitored, whereby such data may be included in a larger database as a record having fields corresponding to parameters and other values of interest. Note that this transfer need not be only one-way, as mentioned above. Rather, it would be possible to download patient information from the mobile device 300 into the patient monitor 100, for example as an initialization step, thereby avoiding the need to manually enter it via a keyboard, or this data could be transferred from the higher-level, remote server 500, in which case the mobile device 300 would be used as an intermediate hub.

In another embodiment, the mobile device 300 may be used together with existing phone communication methods, such as SMS texting to a specific phone number or an e-mail address, so as to add information to the appropriate patient record. The phone number or e-mail address could, for example, be owned by the hospital and assigned to the patient upon registration, or using some other patient ID.

In a scenario in which the healthcare provider is in charge of several patients, a single mobile device may be used to communicate with several monitors, and may distinguish between them using any technology such as user-based monitor selection, GPS positioning, and the simple exchange of information used when the devices first pair with each other or communicate.

In conventional patient monitors, the display 150 is one of the largest, heaviest, and often most fragile components. In one embodiment, assuming that the data transfer rate between the monitor 100 and the mobile device 300 is high enough, it would be possible to remove the monitor display 150 altogether (either as a permanent feature or temporarily, such as in a system in which the display 150 is a plug-in component similar to a secondary monitor for a laptop computer) and instead display the desired information using the built-in display 350 of the mobile device 300. In other words, in such an embodiment, the tasks of the monitor 100 could be to receive and process sensor signals as usual, but the mobile device 300 could take over the display function of the monitor-user interface. Note that although this would require proper display-processing software, such software could either remain within the monitor 100 (with only the video signals being transmitted) or be moved to within the system software 520 or the application-level 540 software of the mobile device 300.

What is claimed is:

1. A data transfer system comprising:
a medical device;
a mobile device; and
wireless connection circuitry in both the medical device and the mobile device enabling wireless data transfer between the medical device and the mobile device;
said mobile device being configured to transfer data wirelessly from the mobile device to the medical device. The system as in claim 1, in which the medical device is a multi-function patient monitor receiving signals from at least one connected patient sensor

3. The system as in claim 1, in which the mobile device is a cellular telephone.

4. The system as in claim 1, in which the mobile device is a tablet computer.

5. The system as in claim 1, in which the wireless connection circuitry is chosen from the group of technologies including Bluetooth, Near Field Communication, radio frequency, infrared, and optical.

6. The system as in claim 1, in which the mobile device includes a microphone and the data includes audio data.

7. The system as in claim 6, in which the audio data includes at least one of voice-to-text converted data, transcription-converted data, and commands.

8. The system as in claim 1, in which the mobile device includes a camera and the data includes image data.

9. The system as in claim 1, which the mobile device includes a camera and the data includes image data, said mobile device being configured to submit the image data to at least one external system for processing.

10. The system as in claim 1, in which the data includes executable medical device code, said medical device, upon receiving the medical device code, loading and executing it.

11. The system as in claim 1, in which the mobile device is configured to receive and display data from the medical device.

12. The system as in claim 1, in which:
the mobile device is connected with a remote system via a network; and
the mobile device is configured to transfer data received from the medical device to the remote system.

13. The system as in claim 12, in which the network is a cellular telephone network.

14. A data transfer system for patient monitoring comprising:
a multi-function patient monitor receiving signals from at least one connected patient sensor;
a mobile device; and
wireless connection circuitry in both the patient monitor and the mobile device enabling wireless data transfer between the patient monitor and the mobile device;
said mobile device being configured to transfer data wirelessly from the mobile device to the patient monitor;
in which:
the wireless connection circuitry is chosen from the group of technologies including Bluetooth, Near Field Communication, radio frequency, infrared, and optical;
the mobile device includes a microphone and the data includes audio-derived data;
the mobile device is connected with an external system via a network, said external system being inaccessible to the patient monitor; and
the mobile device is configured to transfer data between the patient monitor and the remote system.

15. A method for transferring data to and from a medical device, comprising:
pairing the medical device with a mobile device;
transferring the data wirelessly from the mobile device to the medical device.

16. The method as in claim 15, in which the medical device is a multi-function patient monitor receiving signals from at least one connected patient sensor;
said mobile device being configured to access, via a network, at least one external system that is inaccessible to the patient monitor.

17. The method as in claim 15, further comprising pairing the medical device and the mobile device by scanning, using a camera in the mobile device, an optical marker on the medical device.

18. The method as in claim 15, further comprising pairing the medical device and the mobile device by sensing and interpreting an audio signal emitted by the medical device.

19. The method as in claim 15, in which the mobile device is a cellular telephone.

20. The method as in claim 15, in which the mobile device is a tablet computer.

21. The method as in claim 1, in which the pairing is chosen from the group of technologies including Bluetooth, Near Field Communication, radio frequency, infrared, and optical.

22. The method as in claim 15, further comprising a user entering medical device information into the mobile device in audio form via a microphone and transferring the audio information to the medical device.

23. The method as in claim 22, comprising transferring the audio information to the medical device in an audio format, such that the medical device can reproduce the audio information audibly.

24. The method as in claim 22, comprising converting the audio information into textual form before transferring it to the medical device.

25. The method as in claim 22, further comprising converting the audio information into non-audio digital form by submitting the audio information for conversion to the at least one external system.

26. The method as in claim 15, in which the data includes executable medical device code, said medical device, upon receiving the medical device, loading and executing it.

27. The method as in claim 15, in which the data includes medical device control parameters.

28. The method as in claim 15, further comprising receiving data from the medical device into the mobile device.

29. The method as in claim 15, further comprising creating image data with the mobile device and transferring the image data for storage in the medical device.

30. The method as in claim 15, further comprising displaying medical device—generated patient information on a display of the mobile device.

31. The method as in claim 15, further comprising:
connecting the mobile device with a remote system via a network; and
transferring data received from the medical device to the remote system.

32. The method as in claim 31, in which the network is a cellular telephone network.
